(19) United States
(12) Patent Application Publication
(10) Pub. No.: US 2014/0012091 A1
(43) Pub. Date: Jan. 9, 2014

(54) Systems And Methods For Evaluating A Person's Dynamic Wellness

(71) Applicant: David George Reynolds, New York, NY (US)

(72) Inventor: David George Reynolds, New York, NY (US)

(21) Appl. No.: 13/540,712

(22) Filed: Jul. 3, 2012

(51) Int. Cl. A61B 5/00

(52) U.S. Cl. 600/300

(57) ABSTRACT

A system for evaluating the dynamic wellness (vitality) of a person includes a processor, a memory communicatively coupled to the processor, a database stored within the memory, a holome construction module, and a functional zone assessment module. The holome construction module includes instructions stored in the memory, that when executed by the processor, perform the functions of (1) receiving personal data representing one or more physical attributes of the person, and (2) storing the personal data as a holome in the database. The functional zone assessment module includes instructions stored in the memory, that when executed by the processor, perform the function of evaluating dynamics of the holome to determine whether each of one or more somatic systems of the person are in a compensated state.

### BACKGROUND

It is known to individually examine one or more of a person's somatic systems to determine one or more aspects of their health. For example, a health care provider may examine an x-ray image of a person's leg to diagnose a broken leg, or a health care provider many analyze a person's white blood cell count to detect an infection.

Additionally, it is known to determine health insurance premiums, at least in part, by assessing the general population's risk of contracting a disease or other detrimental condition. For example, a 45 year old female's health insurance premium may be determined by assessing the frequency at which 45 year old females have historically contracted a detrimental condition. In some instances, a person's existing or prior physical ailments, such as current high blood pressure or a previous heart attack, are also considered when determining a health insurance premium.

### SUMMARY

In an embodiment, a system for evaluating the dynamic wellness (vitality) of a person includes a processor, a memory communicatively coupled to the processor, a database stored within the memory, a holome construction module, and a functional zone assessment module. The holome construction module includes instructions stored in the memory, that when executed by the processor, perform the functions of (1) receiving personal data representing one or more physical attributes of the person, and (2) storing the personal data as a holome in the database. The functional zone assessment module includes instructions stored in the memory, that when executed by the processor, perform the function of evaluating dynamics of the holome to determine whether each of one or more somatic systems of the person are in a compensated state.

In an embodiment, a method for evaluating, appraising and risk management of the vitality of a person includes the following steps: (1) collecting, in a vitality evaluation computer, personal data representing one or more physical attributes of the person, (2) storing the personal data in a database of the vitality evaluation computer, and (3) evaluating, using a processor of the vitality evaluation computer, the personal data stored in the database to determine whether each of one or more somatic systems of the person are in a compensated state.

In an embodiment, a method for determining a wellness risk management premium for a person includes the following steps: (1) receiving, in a wellness risk management premium determination computer, a wellness score characterizing an improbability that the person will experience a physical ailment, and (2) determining, using a processor of the wellness risk management premium determination computer, the wellness risk management premium for the person at least partially based on the wellness score.

### BRIEF DESCRIPTION OF THE DRAWINGS

FIG. 1 shows one exemplary system for evaluating a person's vitality, according to an embodiment.

FIG. 2 shows one exemplary method for authenticating a user, according to an embodiment.

FIG. 3 shows one exemplary use of the FIG. 1 system, according to an embodiment.

FIG. 4 shows one exemplary method for evaluating a person's, vitality according to an embodiment.

FIG. 5 shows one exemplary method for evaluating personal data to determine a person's functional zone status, according to an embodiment.

FIG. 6 shows one exemplary method for managing a person's wellness care, according to an embodiment.

### DETAILED DESCRIPTION

As discussed above, it is known to individually examine specific somatic systems to obtain information on a person's vitality. While such examination may yield useful information, it has been discovered that valuable information regarding a person's vitality may be determined by examining the interaction of two or more of the person's somatic systems, instead of individually examining one or more the person's somatic systems.

In particular, a person may be considered to have a “holome,” which is a cumulative chronology representing the totality of information pertaining to the person's form, physiology, and function over time. Examples of information included in a person's holome include, but are not limited to, the person's DNA, fingerprints, facial metrics, and cranial architecture, each of which are often collected over time and under varied conditions. The examination of the dynamic biological interaction of a number hierarchical somatic systems in a person's holome, which is sometimes referred to herein as “Sophiomics,” may yield information about the person's dynamic wellness status. The application of this information is sometimes referred to herein as “Applied Sophiomics.”

Each somatic system of a person is generally capable of functioning without detriment, or without dynamic support system over-compensation, up to around one standard deviation from an individual's normal resting value. Thus, under normal conditions, each somatic system is capable of responding to changing physiological demands and returning to normal resting status without detriment. Such capacity to adapt and interact with all other somatic systems and then return to normal resting values may be referred to as “Zone I Adaptability.” Accordingly, a person having a Zone I status is considered to have strong adaptive physiological performance and therefore be of relatively high vitality.

However, a person may be able to obtain an even higher state of vitality than that represented by Zone I status. In particular, an extremely vital person may upon occasion obtain “Zone 0” status, sometimes referred to as the “Optimal Performance Zone” (“O^{P}Zone”), where the person's somatic systems optimally interact with each other, in the context of heightened awareness and perceptual focus. Typically, an individual obtains Zone 0 status by engaging in practices and/or therapies that promote optimal positive interactions amongst the person's somatic systems. The Optimal Performance Zone is considered a state of mind or an altered mental state, beyond Zone I status.

On the other hand, a minor abnormality, such as a minor injury or biochemical imbalance, may cause at least one of the person's somatic systems to be in a compensated state, meaning that the system does not return to its normal resting value after a physiological demand. Such state where at least one of a person's somatic systems is in a compensated state may be referred to as a “Zone II” state. Thus, a person having Zone II status is typically suffering from a functional deficit, for example, such as a referred visceral compensation, muscular trigger point, or ligament strain. However, it is often possible for the person to obtain Zone I status by taking corrective action, such as by resolving the dynamic compensated state.

Furthermore, in some situations, an unresolved dynamic systemic conflict exists where two or more of a person's somatic systems are in an acute or chronic compensated state, such that the systems do not return to their normal resting values after a physiological demand These compensated systems may be adaptively competing for their individual systemic physiological normality, to the detriment of at least one of the person's other somatic systems. Thus, at least one of the person's somatic systems lacks the ability to return to normal functional values without intervention. Accordingly, Zone II status, by definition, is resolvable if no permanent damage has occurred, or if the form, physiology, and function may be returned to normality without compromising a related system.

Zone III status recognizes an acute physiological, immune, or traumatic episode which digresses outside of a normal adaptive range, such that a person's somatic systems may not easily return to near normal state without intervention or significant systemic compromise. Furthermore, a person having Zone III status is typically suffering from a relatively significant abnormality, such as an acute or chronic biochemical insult, an immune crisis, a significant illness, or an injury. The person may therefore benefit from immediate medical treatment to resolve the abnormality Chronic Zone III status thus provides the context in which disease states may manifest, however, a Zone III crisis may be resolvable without residual permanent dysfunction over time.

Thus, valuable information on a person's wellness and vitality may be determined from Sophiomics. For example, as discussed above, a person's relative wellness state, as for example designated by functional Zone 0, I, II, or III status, may be determined by examining the interaction of the person's somatic systems. Additionally, Sophiomics may be applied to optimize a person's wellness and vitality, such as by following Zone protocols corresponding to the person's functional Zone status, to promote and stimulate positive dynamic wellness status change. For example, therapy may be provided to transition the person from Zone III or II status to a Zone I uncompensated state. Discussed below are some examples of systems and methods that may, at least in part, use Applied Sophiomics to evaluate, manage, and/or optimize one or more aspects of a person's vitality.

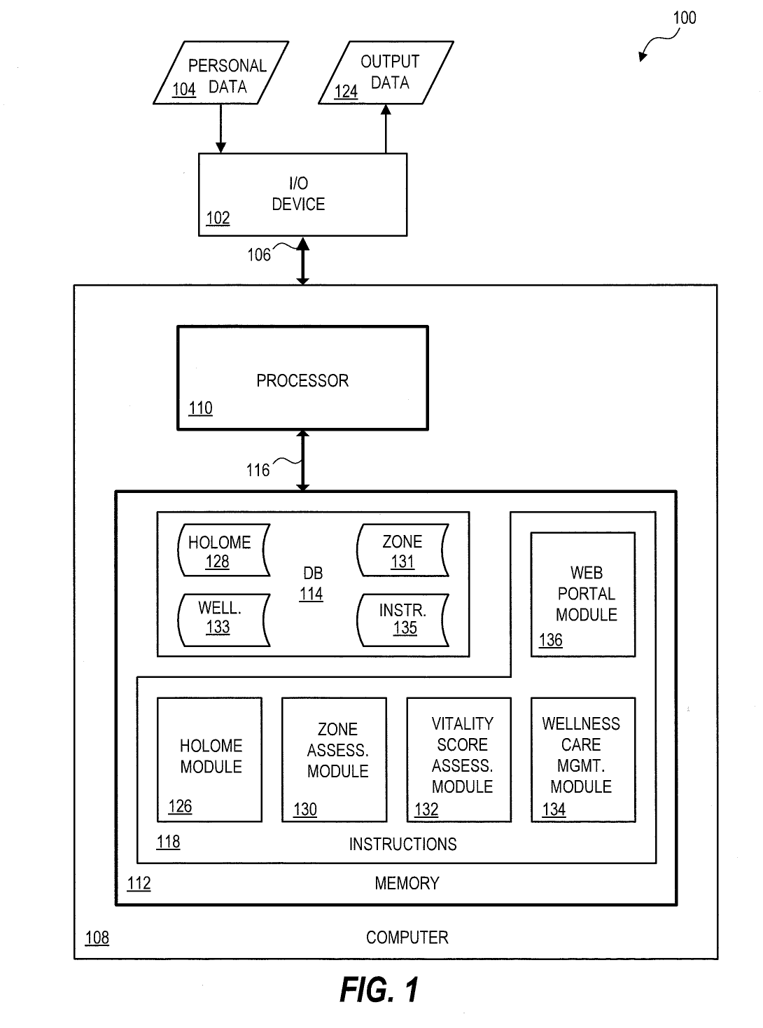

For example, FIG. 1 shows one exemplary system 100 for evaluating a person's vitality. System 100 includes an input/output device 102 for inputting personal data 104 into system 100 and for outputting output data 124 from system 100. Personal data 104 includes data representing one or more physical attributes of a person, such as biometric, biochemical, physiological, and/or genetic data for the person. Examples of output data 124 include, but are not limited to, a person's holome, a person's wellness score, a person's functional zone status, a medical provider referral, a pharmaceutical prescription, and/or instructions to a health care provider or wellness consultant. Examples of input/output device 102 include, but are not limited to, one or more of a keyboard, a mouse, a trackball, an electronic stylus, a microphone, a speaker, a printer, a display, a touch screen, or a computer, such as a personal computer or a smart phone. Input/output device 102 may also include one or more manually or automatically operated biometric diagnostic devices that both collect and input personal data. One example of a possible biometric diagnostic device is an electronic blood pressure monitor that, for example, obtains blood pressure information and automatically inputs it into system 100.

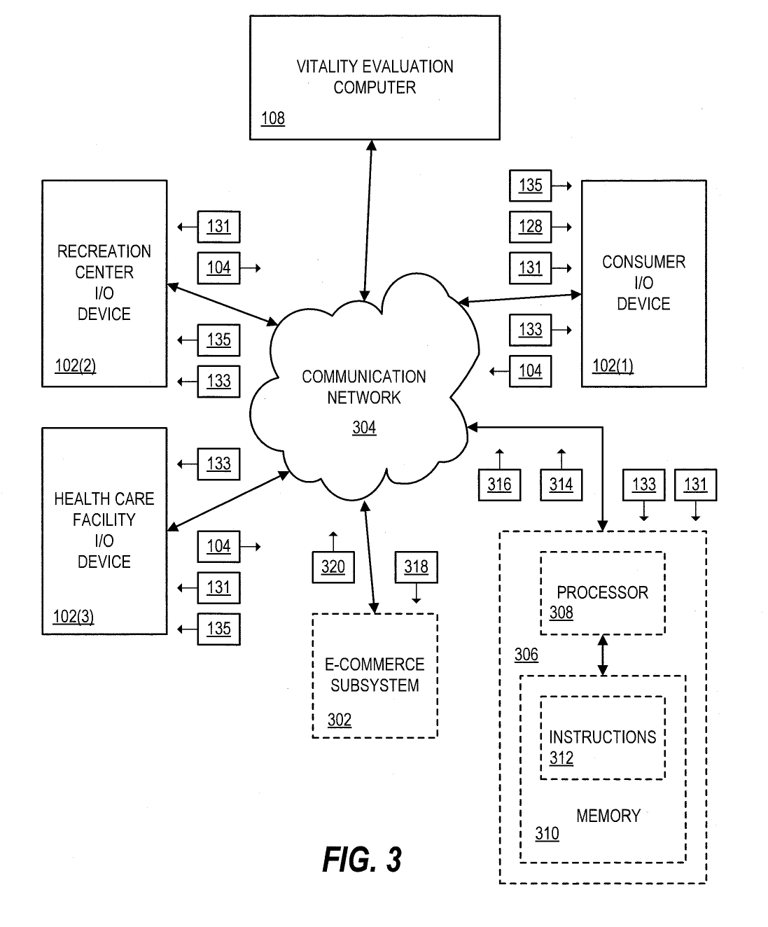

A communication link 106, such as an electrical, optical, and/or wireless communication link, communicatively couples input/output device 102 to a vitality evaluation computer 108. Communication link 106 is at least partially implemented via the Internet in some embodiments. Other configurations of input/output device 102 may be used without departing from the scope hereof. For example, in some alternate embodiments, input/output device 102 is at least partially integrated into vitality evaluation computer 108. As another example, input/output device 102 is replaced with a discrete input device and a discrete output device in some other alternate embodiments. Furthermore, as for example discussed below with respect to FIG. 3, certain embodiments of system 100 include two or more instances of input/output device 102 communicatively coupled to vitality evaluation computer 108.

Vitality evaluation computer 108 includes a processor 110 and memory 112. Memory 112 is, for example, one or more of volatile random access memory, non-volatile random access memory, read only memory, a magnetic data storage disk, an optical data storage disk, and other types of storage medium. A database 114, illustratively shown in memory 112, stores personal data received via input/output device 102. Although database 114 is symbolically shown within memory 112, it is optionally implemented by multiple storage devices, such as multiple magnetic or optical data storage drives, which may or may not be disposed at the same physical location as vitality evaluation computer 108. For example, in certain embodiments, database 114 is implemented by multiple file servers disposed in one or more uniquely located and secure data centers. Database 114 may be encrypted to help protect private information stored therein.

Processor 110 is communicatively coupled to memory 112 via a communication link 116, such as an electrical or optical data bus. Processor 110 executes instructions 118, which are stored in memory 112, to evaluate one or more aspects of a person's vitality from personal data stored in database 114. In some alternate embodiments, instructions 118 are partially or completely stored in database 114. Although processor 110 is shown as a single device, such as a microprocessor, processor 110 may also represent multiple processors configured within a single computer or within multiple computers of a distributed or non-distributed environment.

All components of system 100 may be disposed at a single location, or even in a single enclosure. For example, system 100 could be embodied by a single computer, such as a server, a personal computer, or a smart phone. Alternately, at least some components of system 100 may be disposed at different locations. For example, processor 110 and memory 112 could be located in a data center, while input/output device 102 is located at a remote site where personal data 104 is collected. Possible examples of such remote site include, but are not limited to, a health care facility and a recreation facility.

In certain embodiments, instructions 118 include a holome construction module 126. The instructions of module 126, when executed by processor 110, perform the functions of constructing and/or maintaining respective holomes 128 for one or more people based on personal data 104. Module 126 is typically configured to store holomes 128, or specific parts thereof, in database 114 as shown, although module 126 may alternately or additionally be configured to export holomes 128 as output data 124 via input/output device 102. In some embodiments, module 126, when executed, controls input/output device 102 to collect personal data 104 needed to construct and/or maintain holomes 128 within database 114. For example, in some embodiments, input/output device 102 includes one or more automated biometric diagnostic devices, and module 126 controls such devices to automatically collect and input personal data 104 used to construct or update holome 128. Additionally, module 126 may also control input/output device 102 to generate instructions for collecting personal data 104 used to create and/or maintain holome 128. For example, if updated height and weight information is needed to update holome 128 so that it is a cumulative chronology, module 126 and input/output device 102 cooperate to display instructions to measure and input the person's certified height and weight. A health care professional then measures the person's height and weight and enters the information as personal data 104 via input/output device 102, wherein module 126 stores the information within holome 128.

Instructions 118 optionally include one or more modules in addition to or in place of module 126. For example, some embodiments include one or more of a functional Zone assessment module 130, a vitality or wellness score assessment module 132, a dynamic wellness management module 134, and/or a web portal module 136. Functional Zone assessment module 130, when executed by processor 110, associates the person with a dynamic functional Zone status, e.g., Zone I, Zone II, or Zone III, from personal data 104 stored in database 114. For example, in some embodiments, module 130 is operable to determine a person's functional Zone assessment 131 from dynamics of their respective holome 128 stored in database 114, such as by evaluating holome 128 to determine whether each of one or more somatic systems of the person are in a compensated state. As another example, in some embodiments, module 130 is operable to determine a person's functional Zone assessment 131 at least partially from relationships between compensated somatic systems. Module 130 is typically configured to store functional Zone assessments 131 in database 114 as shown, although module 130 may alternately or additionally be configured to export Zone assessments 131 as output data 124 via input/output device 102.

Wellness score assessment module 132, on the other hand, when executed by processor 110, determines a person's wellness score 133, which characterizes the improbability that a person will experience a physical ailment, such as one or more particular physical ailments. Thus, a person's wellness score 133 reflects what is “right” with the person, instead of what is “wrong” with the person. The wellness score 133 could be monetized. For example, wellness score 133 could be used to establish a wellness risk management or health insurance discount program, where people with higher wellness scores (indicating that the people are relatively vital), pay less for wellness risk management or health insurance than people with lower wellness scores. Module 132 determines a wellness score 133 at least partially from personal data 104 stored in database 114. Module 132 is typically configured to store wellness score 133 in database 114 as shown, although module 132 may alternately or additionally be configured to export wellness score 133 as output data 124 via input/output device 102. Furthermore, in some embodiments, module 132 at least partially determines a wellness risk management premium or health insurance discount for the person based at least partially on the person's wellness score 133.

Wellness care management module 134, when executed by processor 110, manages a person's wellness care by evaluating personal data stored in database 114 and determining what optimal and optional action to take based, at least in part, on the personal data. For example, in certain embodiments, module 134 cooperates with Zone assessment module 130 to follow functional Zone protocols corresponding to a person's functional Zone assessment, to promote and stimulate positive vitality status change. Such protocols include, for instance, specifying, in the form of wellness care instructions 135, appropriate therapy for transitioning the person towards Zone I status. As another example, in some embodiments, module 134 cooperates with Zone assessment module 130 to evaluate holome 128 stored in database 114 and generate wellness care instructions 135 based at least partially on the personal data. In such embodiments, module 130 evaluates the personal data stored in holome 128 to determine the person's functional Zone status, and module 134 then generates wellness care instructions 135 based at least partially on zone protocols for the functional Zone. Module 134 is typically configured to store instructions 135 in database 114 as shown, although module 134 may alternately or additionally be configured to export instructions 135 as output data 124 via input/output device 102.

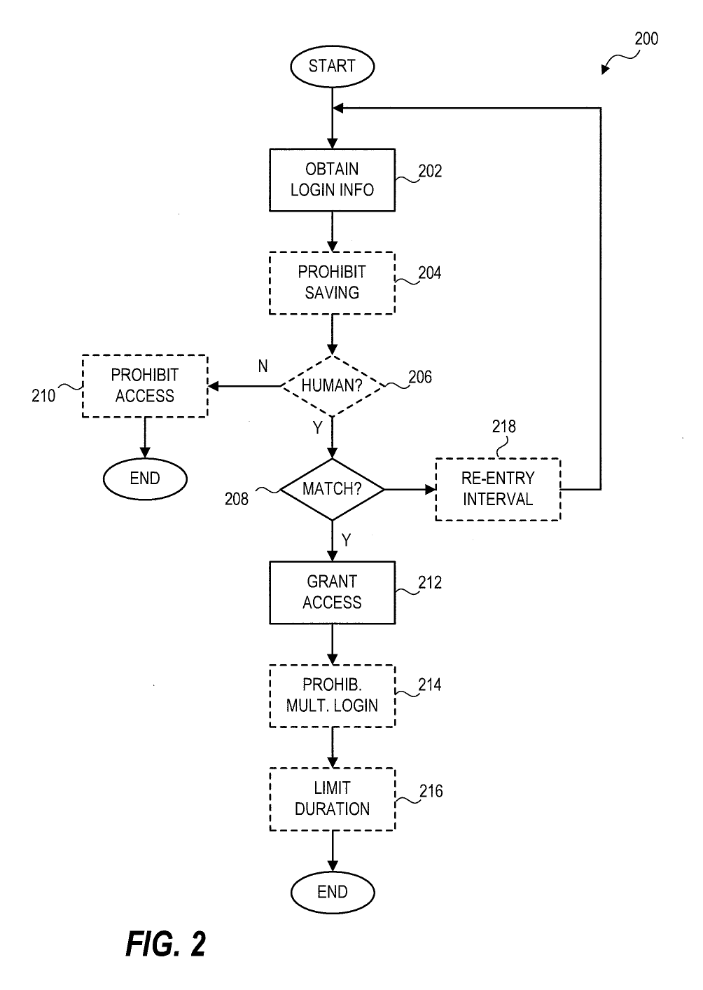

Web portal module 136, when executed by processor 110, interfaces system 100 with the World Wide Web (“web”). For example, some embodiments of module 136, when executed by processor 110, perform an exemplary user authentication method 200 shown in FIG. 2. Method 200 begins with a step 202 of obtaining user login information, including biometric authentication information. In some embodiments, the login information also includes a user identification code and/or a user password. In one example of step 202, module 136 controls input/output device 102 to receive user identification, password, and biometric information as encrypted personal data 104. Optional step 204 prohibits the user from saving their password and/or other login information in their web client, thereby promoting system security. In one example of this step, module 136 controls input/output device 102 to send instructions to the user's web client preventing password saving. Optional decision step 206 determines whether the user is a human. If so, the method proceeds to decision step 208. If not, the method proceeds to optional step 210 where access is denied, and the method subsequently ends. In one example of step 206, module 136 controls input/output device 102 to output a series of characters recognizable by a human but not by a machine. Module 136 then controls input/output device 102 to prompt the user to input the outputted character series via input/output device 102, and module 136 compares the inputted character series to the outputted character series. If the two character series match, module 136 assumes the user is a human; otherwise, module 136 assumes the user is a machine. One example of step 210 is module 136 prohibiting access to database 114.

Decision step 208 compares the login information obtained in step 202 to unique identifying holomic information associated with the user. One example of step 208 is module 136 comparing the login information obtained in step 202 to a known unique identifier (not shown) stored in hashed form within database 114 to determine whether the login information matches the stored information. If the information matches, the user is granted access in step 212. One example of step 212 is module 136 granting the user access to their holome 128 stored in database 114. Method 200 optionally further includes step 214 of preventing multiple simultaneous logins by the same user and/or step 216 of limiting the duration of a user's access. As one example of these steps, module 136 may control database 114 to prevent a user from accessing database 114 via multiple simultaneous sessions and/or to limit the duration of the user's access to database 114.

If the information is determined to not match in decision step 208, the method returns to step 202, optionally via reentry interval step 218. Reentry interval step 218 increases the minimum time between login attempts after an unsuccessful login, thereby discouraging password guessing attempts. One example of step 218 is module 136 rejecting login information for minimum time duration after a failed login attempt.

FIG. 3 shows one exemplary use of system 100 in an application including a plurality of input/output devices 102, an optional electronic commerce (“e-commerce”) subsystem 302, and an optional wellness risk management premium determination computer 306. A communication network 304 communicatively couples input/output devices 102, optional e-commerce subsystem 302, and optional computer 306 to vitality evaluation computer 108. Communication network 304 includes, for example, the Internet, a cellular telephone network, a wide area network, and/or a local area network. Although system 300 is shown as including three input/output devices 102, the number and configuration of input/output devices 102 may vary without departing from the scope hereof.

A personal input/output device 102(1) is communicatively coupled to vitality evaluation computer 108 via communication network 304. Personal input/output device 102(1) is used, for example, by a subscriber to system 100. Personal input/output device 102(1) allows a person to (1) access his or her holome 128 stored in database 114, (2) access vitality evaluation information determined by computer 108, such as functional Zone assessment 131 or wellness score 133, and/or (3) input personal data 104 into computer 108. For example, in some embodiments, wellness care management module 134 controls personal input/output device 102(1) to output functional Zone assessment 131 and/or wellness care instructions 135 based on the functional Zone assessment. As another example, in certain embodiments, holome construction module 126 controls personal input/output device 102(1) to prompt a person to input personal data 104 needed to supplement the person's holome 128. Module 126 then obtains the inputted data from personal input/output device 102(1) and updates holome 128 accordingly. Personal input/output device 102(1) includes, for example, one or more of a personal computer, a smart phone, or a communication device intended primarily or solely for use with system 100.

Communication network 304 also communicatively couples a recreation center input/output device 102(2) to vitality evaluation computer 108. Recreation center input/output device 102(2) is disposed at a recreation center or similar place where people engage in physical activity and is used, for example, by recreation center patrons and/or recreation center employees. In some embodiments, biometric tests are conducted at the recreation center, and the test results are inputted as personal data 104 into vitality evaluation computer 108 via recreation center input/output device 102(2). For example, in certain embodiments, recreation center input/output device 102(2) includes exercise equipment having automatic biometric diagnostic capability, and holome construction module 126 controls the exercise equipment to collect biometric data while a user exercises. Additionally, in some embodiments, wellness care management module 134 controls recreation center input/output device 102(2) to output vitality evaluation information, such as a person's functional Zone assessment 131 and/or wellness score 133. In these embodiments, wellness care management module 134 optionally also controls input/output device 102(2) to output wellness care instructions 135. For example, in the case where a recreation center patron has a Zone II status, module 134 may control recreation center input/output device 102(2) to provide the patron, and/or a recreation center worker, suggestions on how to obtain Zone I status in the form of wellness care instructions 135.

A health care facility input/output device 102(3) is also communicatively coupled to vitality evaluation computer 108 by communication network 304. Input/output device 102(3) is disposed at a location where health care is provided, such as at a health care clinic or at a hospital. Input/output device 102(3) is used, for example, by patients and/or health care workers at the health care facility. Personal data 104 needed to construct holome 128 is, for example, inputted into computer 108 via input/output device 102(3) in some embodiments. For example, health care facility input/output device 102(3) may include health care equipment with automatic biometric diagnostic capability, and holome construction module 126 controls the health care equipment to collect biometric data needed for holome 128. Moreover, in certain embodiments, wellness care management module 134 controls input/output device 102(3) to output information on a person's dynamic wellness, such as their functional Zone assessment 131 or wellness score 133. Furthermore, in some embodiments, module 134 controls input/output device 102(3) to output wellness care instructions 135 based on a person's functional Zone assessment.

As discussed above, communication network 304 communicatively couples optional wellness risk management premium determination computer 306 to health evaluation computer 108 in some embodiments. Computer 306 includes a processor 308 and a memory 310 communicatively coupled to processor 308. Processor 308 executes instructions 312 stored in memory 310 to determine a person's wellness risk management premium at least partially from vitality information, such as wellness score 133 information, received from computer 108. For example, in some embodiments, instructions 312, when executed by processor 308, set at least a portion of the wellness risk management premium to be inversely proportional to wellness score 133 received from computer 108. As another example, in some other embodiments, instructions 312, when executed by processor 308, determine, in part, a health insurance premium discount based on wellness score 133 received from computer 108. Computer 306 optionally sends to vitality evaluation computer 108 requests 314 for vitality information, such as wellness score 133 and/or functional Zone assessment 131 information.

Computer 306 optionally outputs wellness risk management information 316, such a wellness risk management premium amount or a health insurance premium discount, to vitality evaluation computer 108 and/or to an input/output device 102. For example, in some embodiments, computer 306 outputs wellness risk management information 316 directly to personal input/output device 102(1) via communication network 304. Additionally, in some embodiments, wellness care management module 134 is operable to interact with computer 306 via communication network 304 to enroll a user in a wellness risk management program or health insurance program. Additionally, in certain embodiments, a user may interact directly with computer 306 via an input/output device 102 to enroll in a wellness risk management program or a health insurance program.

Although wellness risk management premium determination computer 306 is symbolically shown as a single device, it is optionally embodied by a number of different subsystems, which are not necessarily disposed at the same physical location. For example, computer 306 could be implemented via a distributed computer system. Wellness risk management premium determination computer 306 is also integrated in vitality evaluation computer 108 in some alternate embodiments.

Furthermore, in some alternate embodiments, vitality evaluation computer 108 determines wellness risk management premium information, such as a wellness risk management premium amount or a health insurance premium discount, from personal data 104 stored in database 114. For example, in some embodiments, instructions 118 include a wellness risk management premium module (not shown) that when executed by processor 110, determines a wellness risk management premium or a health insurance premium discount from wellness score 133 stored in database 114.

In certain embodiments, system 100 is adapted such that dynamic wellness evaluation information and/or dynamic wellness evaluation services may be purchased from system 100. For example, in some embodiments, a person may subscribe to system 100 for a period of time, such as to obtain functional Zone assessment services and/or wellness care instructions during this time period. As another example, in certain embodiments, a party, such as an insurance company, purchases vitality information for one or more people from system 100. Accordingly, some embodiments of system 100 include e-commerce subsystem 302, as shown in FIG. 3. E-commerce subsystem 302 is adapted to facilitate purchase of information and/or services from system 100.

E-commerce subsystem 302 receives a party's payment information 318. In some embodiments, payment information 318 is transferred directly from an input/output device 102 to e-commerce subsystem 302. In other embodiments, payment information 318 is transferred to e-commerce subsystem 302 via vitality evaluation computer 108. Payment information 318 includes, for example, credit or debit card information, financial account information, purchase order information, electronic funds transfer information, and/or electronic commerce information. Subsystem 302 processes payment information 318, and if payment information 318 is valid, outputs a payment acknowledgement 320 to vitality evaluation computer 108. Payment acknowledgement 320 indicates valid payment. Accordingly, some embodiments of system 100 condition access to system 100's information and/or services on receiving payment acknowledgment 320.

For example, in some embodiments, system 100 is adapted such that a person who wishes to subscribe to system 100 may input payment information 318 via personal input/output device 102(1). The person's payment information 318 is transmitted to e-commerce subsystem 302 either directly via communication network 304, or via vitality evaluation computer 108 and network 304. E-commerce subsystem 302 processes the person's payment information 318, and if the payment information is valid, subsystem 302 outputs payment acknowledgement 320 to vitality evaluation computer 108. Vitality evaluation computer 108 grants the person access to system 100's services and data for a specified time period, for example, after receipt of payment acknowledgement 320.

As another example, in some embodiments, system 100 is adapted such that an insurance company which wishes to purchase a number of wellness scores may output a request 314 for wellness scores and associated payment information 318 to vitality evaluation computer 108. Vitality evaluation computer 108 forwards the insurance company's payment information 318 to e-commerce subsystem 302. E-commerce subsystem 302 evaluates the insurance company's payment information 318, and if valid, outputs a payment acknowledgement 320 to vitality evaluation computer 108. Vitality evaluation computer 108 transfers wellness scores 133 identified by request 314 after receiving payment acknowledgement 320.

Although e-commerce subsystem 302 is shown as being separate from vitality evaluation computer 108, in some alternate embodiments, e-commerce subsystem 302 is integrated in computer 108. Furthermore, although sale of wellness information and/or services is discussed with respect to e-commerce subsystem 302, system 100 could be adapted to cooperate with alternative electronic commerce subsystems without departing from the scope hereof.

In some embodiments, access to vitality evaluation computer 108 and/or information associated therewith is controlled, at least partially, by biometric authentication. For example, in certain embodiments, a person must provide biometric authentication information to vitality evaluation computer 108 to access his or her holome 128 via personal input/output device 102(1). In this embodiment, computer 108 grants the person access to his or her holome 128 only if biometric authentication information provided by the person matches corresponding data of holome 128. As another example, in certain embodiments, a health care worker may access functional Zone assessment 131 information via health care facility input/output device 102(3) only upon providing valid biometric authentication information to vitality evaluation computer 108.

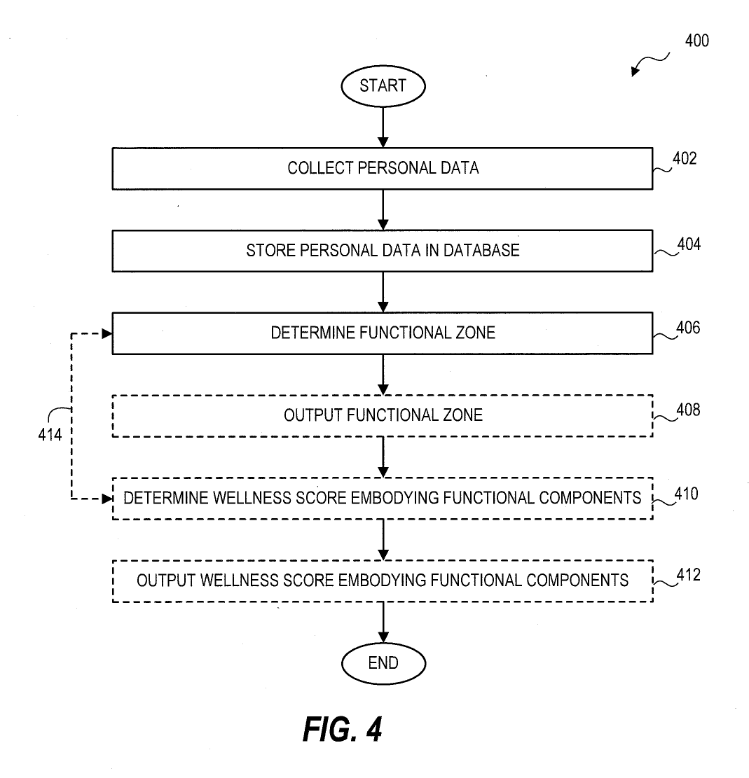

FIG. 4 shows an exemplary method 400 for evaluating vitality of a person. Method 400 is executed, for example, by one or more of modules 126, 130, and 132 of system 100. Method 400 begins with step 402 of collecting personal data representing one or more physical attributes of the person, such as biometric, biochemical, physiological, and/or genetic data for the person. In one example of step 402, holome construction module 126 controls input/output device 102 to obtain personal data, such as from biometric diagnostic equipment, and inputs the data into vitality evaluation computer 108 (FIG. 1). Personal data is collected in step 402, for example, at two or more different times and/or under two or more different conditions. In step 404, the personal data is stored in a database. In one example of step 404, module 126 controls database 114 to store the data collected in step 402 as holome 128.

In step 406, the personal data stored in the database is evaluated to associate the person with a corresponding functional Zone. In one example of step 406, Zone assessment module 130 evaluates dynamics of the person's holome 128 stored in database 114 to determine the corresponding functional Zone assessment 131 for the person, such as by determining which somatic systems are in a compensated state. For example, if module 130 determines from holome 128 that zero, only one, or two or more somatic systems are in a compensated state, module 130 respectively associates the person with Zone I, Zone II, or Zone III. The functional Zone assessment 131 from step 406 is outputted in optional step 408. In one example of step 408, Zone assessment module 130 controls input/output device 102 to output functional zone assessment 131 as output data 124.

Method 400 optionally further includes steps 410 and 412. Step 410 determines the person's wellness score 133, which optionally embodies functional components, as discussed below, from the personal data stored in the database. Step 412 outputs the wellness score. In one example of step 410, wellness score assessment module 132 determines the person's wellness score from their functional zone determination from step 406. In one example of step 412, module 132 controls input/output device 102 to output wellness score 133 as output data 124. In some embodiments, functional zone assessment 131 is determined at least partially from or in conjunction with wellness score 133, as indicated by arrow 414 showing reciprocation between steps 406 and 410.

In certain embodiments, wellness score 133 is derived from algorithmic combination of two or more functional components. One example of a possible functional component is an Autonomic Index, which is the quotient of Heart Rate Variability divided by Systemic Sympathetic Tone. Another possible functional component is Intrinsic Core Stability, which is the quotient of xBMI divided by Positional Leg Load Resistance, where xBMI is a variation of Body Mass Index that integrates bone size, age, sex, and activity information. Retinal Perceptual Void Analysis data, which can be used as an indicator of brain stem functional integration and corresponds to the functional condition of a person's integrated cranial nerve neurology at a given time, is yet another example of a possible functional component. A Retinal Perceptual Void Analysis is performed, for example, by a process including comparing left and right retinal perceptual void areas, eccentricities, vertical shift, lateral shift, and/or tilts, in a retinal perceptual void map.

In some embodiments, each functional component is indexed to a respective norm for a particular population group before algorithmic combination. In some other embodiments, the functional component is indexed to a norm of the average for a particular population group. The following is one exemplary expression for deriving wellness score 133:

Vitality Score=(Personal Score)/(Population Norm) EQN. 1

where Vitality Score is equal to or proportional to wellness score 133, Personal Score is the algorithmic combination of two or more functional components for a person, and Population Norm is the norm of the average of such functional components for an appropriate population group.

As discussed above, a person's wellness score 133 measures and correlates the improbability that the person will experience a physical ailment. One possible use of wellness score 133 is to at least partially determine an individual's wellness risk management premium. In particular, a high wellness score 133 indicates that the person is relatively unlikely to contract a detrimental outcome; accordingly, at least a portion of a person's wellness risk management premium may be set in inverse proportion to their wellness score. Thus, people who optimize and maintain their natural good health through beneficial lifestyle choices and behaviors pay less for wellness risk management than people who do not, assuming all else is equal. In contrast, people who practice beneficial lifestyle choices and behaviors pay a disproportionate cost under traditional health insurance models because such models do not take into account a person's wellness. Thus, in some embodiments, step 412 of the outputting wellness score 133 further includes transmitting wellness score 133 to an external system adapted to determine a health insurance premium, such as discussed above with respect to wellness risk management premium determination computer 306 in FIG. 3.

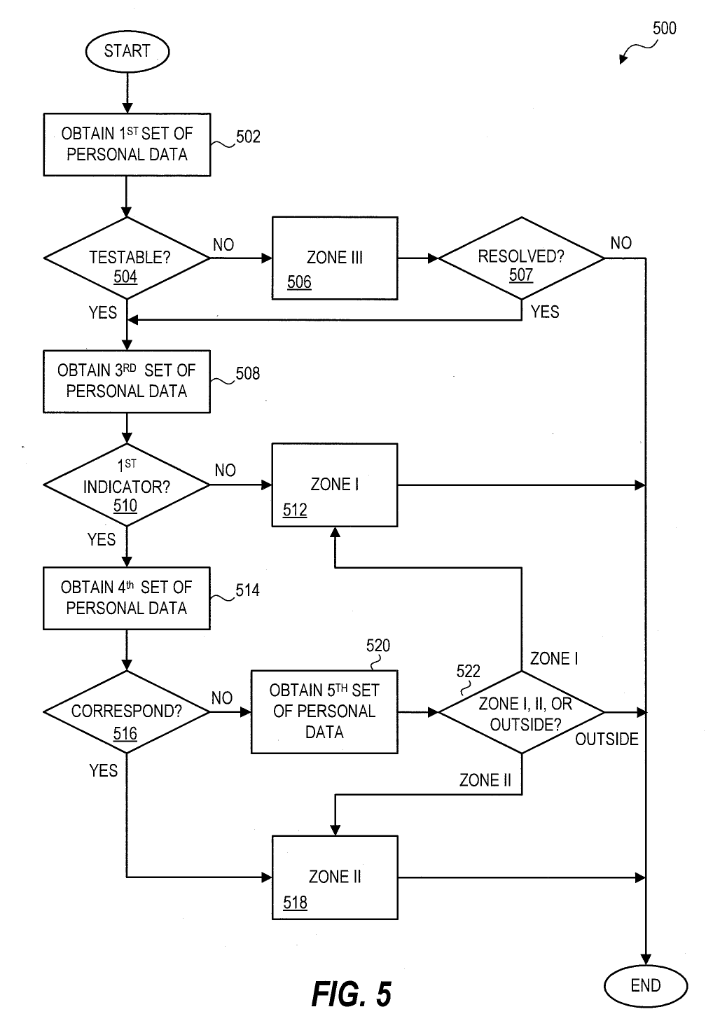

FIG. 5 shows one exemplary method 500 for evaluating personal data to determine a person's functional Zone status. Method 500 is executed, for example, by one or both of modules 126 and 130 of system 100. Method 500 begins with step 502 of obtaining a first set of personal data representing one or more physical attributes of the person. In one example of step 502, holome construction module 126 controls input/output device 102 to obtain a first set of personal data, such as neuromuscular, biometric, and biochemical data, and module 126 controls database 114 to store the data in holome 128. In decision step 504, the first set of personal data is evaluated to determine whether the person's body is adaptively responsive to the functional testing method, that is, whether the person is able to be tested using Applied Sophiomic Protocols. In one example of decision step 504, module 130 evaluates data stored in holome 128 to determine whether the person is able to be tested using Applied Sophiomic Protocols.

If decision step 504 determines that the person is not adaptively responsive to the functional testing method, the person is determined to have a Zone III functional Zone status, and Zone III protocols are followed in step 506. Step 506 also includes obtaining a second set of personal data after following the Zone III protocols. In one example of step 506, module 130 controls input/output device 102 to output a Zone III functional Zone assessment 131 and Zone III protocols as output data 124, module 126 controls input/output device 102 to obtain additional personal data, and module 126 controls database 114 to store the additional personal data in holome 128. Decision step 507 evaluates the second set of personal data to determine whether the person is now able to be tested using Applied Sophiomic Protocols. If so, method 500 continues with step 508; otherwise method 500 ends. In one example of decision step 507, module 130 evaluates additional data stored in holome 128 to determine whether the person is able to be tested using Applied Sophiomic Protocols.

If the person's body is able to be tested, method 500 continues with step 508. In step 508, a third set of personal data is obtained. In one example of step 508, holome construction module 126 controls input/output device 102 to obtain a third set of personal data, such as pelvic test data, and module 126 controls database 114 to store the data in holome 128. Decision step 510 evaluates the data to determine whether there is a first physical indicator, such as a pelvic neuromuscular indicator. In one example of step 510, Zone assessment module 130 evaluates pelvic data stored in holome 128 to determine whether there is a pelvic neuromuscular indicator; if not, and if Zone I indicators are present, the method proceeds to step 512 where the person is determined to have a Zone I functional Zone assessment 131, and the method ends. In one example of step 512, zone assessment module 130 controls input/output device 102 to output a Zone I functional Zone assessment 131 as output data 124.

On the other hand, if a first physical indicator is identified in decision step 510, the method proceeds to step 514 where a fourth set of personal data providing a second physical indicator is obtained. In one example of step 514, holome construction module 126 controls input/output device 102 to obtain a fourth set of personal data, such as cranial data providing a cranial indicator, and module 126 controls database 114 to store the data in holome 128. Decision step 516 evaluates the fourth set of personal data to determine whether the second physical indicator corresponds to the first physical indicator. If so, the person is determined to have a Zone II functional Zone assessment 131 in step 518, and the method ends. An example of steps 516 and 518 is zone assessment module 130 evaluating cranial personal data stored in holome 128 and determining that a cranial indicator corresponds to a pelvic indicator, and module 130 then controlling input/output device 102 to output a functional Zone II functional Zone assessment 131 as output data 124.

If decision step 516 instead determines that the second physical indicator does not correspond to the first physical indicator, method 500 proceeds to step 520 where a fifth set of personal data is obtained. The fifth set of personal data is evaluated in decision step 522 to determine whether a Zone I or a Zone II functional Zone assessment 131 is warranted, or whether the data indicates a course of action outside the scope of method 500 so that the method ends. If Zone I or II status is warranted, method 500 respectively proceeds to step 512 or 518, otherwise, the method ends. In one example of step 520, holome construction module 126 controls input/output device 102 to obtain a fifth set of personal data, such as somato/visceral and spino/pelvic data, and module 126 controls database 114 to store the data in holome 128. In one example of step 522, zone assessment module 130 evaluates somato/visceral and spino/pelvic data stored in holome 128 and determines whether Zone I or Zone II functional Zone assessment 131 is appropriate based on the data. In another example of step 522, zone assessment module 130 evaluates data stored in holome 128 to determine that while Zone II indicators are present, a chronic condition such as a low grade dental infection may be the underlying cause of somatic system compensations, such that a course of action is outside the scope of method 500, and the method ends.

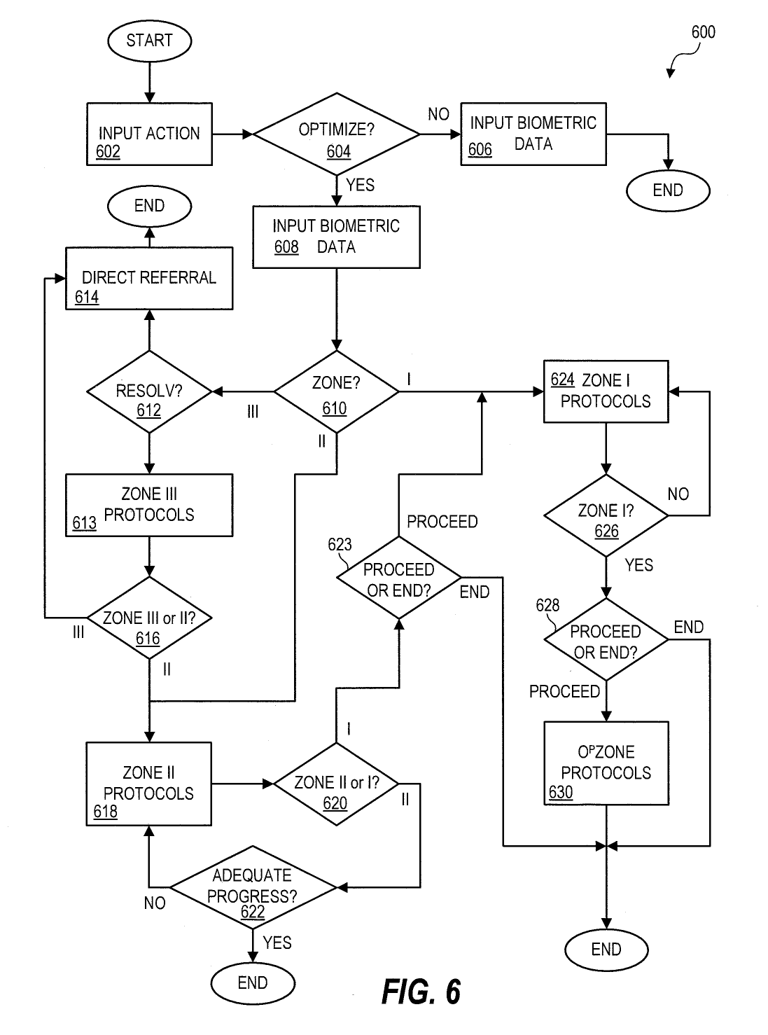

FIG. 6 shows an exemplary method 600 for managing a person's wellness care, such as at a facility purposefully built to execute method 600, a health care facility, or a recreation facility. Method 600 is executed, for example, by one or more of modules 126, 130, and 134 of system 100, and method 600 may be used, for example, to help optimize a person's vitality. The method begins with step 602 of inputting information indicating the action to be performed. Possible actions include, but are not limited to, routine biometric data collection, such as to update the person's holome, or performance or special biometrics, such as to document, evaluate, and to improve the person's vitality. In one example of this step, wellness care management module 134 of system 100 (FIG. 1) controls input/output device 102 to input the action to be performed as personal data 104. Decision step 604 evaluates the action to determine whether the person's wellness is to be optimized. If not, method 600 proceeds to step 606 where biometric data is inputted and stored, and the method ends. In one example of step 604, wellness care management module 134 evaluates data received via step 602 to determine whether the person's wellness is to be optimized. In one example of step 606, in turn, module 134 controls automatic biometric diagnostic equipment to obtain and input biometric data via input/output device 102, and module 134 controls database 114 to store the data in holome 128.

On the other hand, if the person's wellness is to be optimized, method 600 proceeds from decision step 604 to step 608 where biometric data is inputted. In one example of step 608, module 134 controls input/output device 102 to input biometric data as personal data 104 via input/output device 102, and module 134 controls database 114 to store the data in holome 128. In step 610, the person's functional Zone assessment 131 is determined. In one possible example of step 610, zone assessment module 130 evaluates the biometric data inputted in step 608 to determine how many, if any, somatic systems are in a compensated state.

If step 610 determines that the person has Zone III status, the method proceeds to decision step 612 to determine whether the Zone III state is resolvable through Zone III protocols, or whether the Zone III state requires a direct referral to a medical provider. If the Zone III state is resolvable, the method proceeds to Zone III protocols 613, otherwise, the method proceeds to direct referral step 614. In one example of step 612, wellness care management module 134 evaluates the biometric data inputted in step 608 to determine whether the Zone III state is resolvable through Zone III protocols. In one example of step 613, wellness care management module 134 controls input/output device 102 to generate instructions 135 to a health care provider to provide corrective therapy to resolve the issue or issues resulting in Zone III status. Step 614 is performed, for example, by module 134 controlling input/output device 102 to provide the person a health care referral as output data 124.

Method 600 includes decision step 616 of collecting and evaluating additional personal data to determine whether the person has Zone III or Zone II status after performing the Zone III protocols. If the person still has Zone III status, method 600 proceeds to direct referral step 614 and subsequently ends. If the person now has Zone II status, the method proceeds to Zone II protocols 618. An example of step 616 is wellness care management module 134 controlling input/output device 102 to receive additional personal data 104, module 134 controlling database 114 to store the additional data in holome 128, and module 134 evaluating the additional data in holome 128 to determine whether the person has a Zone II or III functional Zone assessment 131.

If step 610 determines that the person has a Zone II functional Zone assessment 131, method 600 proceeds from step 610 to Zone II protocols in step 618. Zone II protocols typically include corrective therapy to attempt to improve the person's vitality such that they achieve Zone I (or better) status. An example of step 618 is wellness care management module 134 controlling input/output device 102 to generate instructions 135 to a health care provider for providing Zone II appropriate therapy. In decision step 620, additional personal data is collected and evaluated to determine whether the person still has Zone II status, or whether the Zone II protocols enabled the person to obtain Zone I status. If the person still has Zone II status, method 600 proceeds to decision step 622 where it is determined whether the Zone II protocols were effective, i.e., whether adequate progress towards Zone II compensation resolution has been obtained. If not, method 600 returns to step 618 for additional Zone II protocols, otherwise, method 600 ends. In one example of step 620, wellness care management module 134 controls input/output device 102 to receive additional personal data 104, module 134 controls database 114 to store the additional personal data in holome 128, and module 134 evaluates the additional personal data in holome 128 to determine whether the person has a Zone II or Zone I functional Zone assessment 131. In one example of step 622, module 134 compares recent and older personal data in holome 128 to determine whether the Zone II protocols improved the person's vitality. If decision step 620 determines that the person has Zone I status, method 600 proceeds to decision step 623, which determines whether to proceed with Zone I protocols, or to end. If decision step 623 determines to proceed with Zone I protocols, the method proceeds to Zone I protocols 624, otherwise, the method ends. One example of decision step 623 is module 134 obtaining from a user, via input/output device 102, instructions on whether to proceed with Zone I protocols or to end. Method 600 also reaches Zone I protocols 624 from step 610 if step 610 determines that the person has Zone I status.

Zone I protocols include techniques, practices, and/or adaptive training methods to try to move the person beyond Zone I status. An example of step 624 is wellness care management module 134 controlling input/output device 102 to generate instructions 135 to a health care provider for providing Zone I appropriate therapies. In step 626, additional personal data is collected and evaluated to confirm that the person has Zone I status, and whether the Zone I protocols caused the person to move beyond Zone I status. In one example of step 626, wellness care management module 134 controls input/output device 102 to receive additional personal data 104, module 134 controls database 114 to store the additional personal data in holome 128, and module 134 evaluates the additional personal data in holome 128 to determine whether the person continues to have Zone I status and has moved toward the Optimal Performance Zone (O^{P}Zone). In one example of step 626, module 134 compares recent and older personal data in holome 128 to determine whether the Zone I protocols equaled or improved the person's wellness biomarkers, such as the person's wellness score. If so, method 600 continues to decision step 628, otherwise, method 600 returns to step 624 or alternatively ends.

Decision step 628 determines whether to proceed to Optimal Performance Zone protocols. If so, method 600 proceeds to O^{P}Zone protocols 630, otherwise, the method ends. One example of decision step 628 is module 134 obtaining from a user, via input/output device 102, instructions on whether to proceed beyond Zone I protocols or to end. One example of step 630 is wellness care management module 134 controlling input/output device 102 to generate instructions 135 to a health care provider for providing O^{P}Zone appropriate therapies.

As discussed above, functional Zone protocols may be followed to promote and stimulate positive wellness status change, thereby helping transition a person to a lower numbered functional Zone status, such as from Zone III or Zone II status towards Zone I (or even O^{P}Zone) status. In some embodiments, functional zone protocols include one or more therapies which identify dysfunctional elements inhibiting optimal vitality, and apply strategies to address and resolve the dysfunction. For instance, a functional Zone protocol may include identifying and resolving conflicts between a person's somatic systems, thereby promoting positive vitality status change over time. Some examples of possible therapies include, but are not limited to, Cranio-Skeletal, Oral Appliance Therapy, and Biochemical Balancing, each of which promote positive vitality status change.

Combinations of Features

Features described above as well as those claimed below may be combined in various ways without departing from the scope hereof. The following examples illustrate some possible combinations:

(A1) A system for evaluating dynamic wellness (vitality) of a person may include a processor, a memory communicatively coupled to the processor, a database stored within the memory, a holome construction module, and a functional zone assessment module. The holome construction module may include instructions stored in the memory, that when executed by the processor, perform the functions of (1) receiving personal data representing one or more physical attributes of the person, and (2) storing the personal data as a holome in the database. The functional zone assessment module may include instructions stored in the memory, that when executed by the processor, perform the function of evaluating dynamics of the holome to determine whether each of one or more somatic systems of the person are in a compensated state.

(A2) In the system denoted as (A1), the functional zone assessment module may further include instructions stored in the memory, that when executed by the processor, perform the function of associating one of a plurality of different functional zones with the person based at least partially on a relationship between somatic systems of the person that are in a compensated state. Each of the plurality of different functional zones may represent a different relative wellness state of the person.

(A3) In the system denoted as (A2), the functional zone assessment module may further include instructions stored in the memory, that when executed by the processor, perform the function of associating the person with a first of the plurality of different functional zones if each of the one or more somatic systems of the person are in an uncompensated state.

(A4) In either of the systems denoted as (A2) or (A3), the functional zone assessment module may further include instructions stored in the memory, that when executed by the processor, perform the function of associating the person with a second of the plurality of different functional zones if only one of the one or more somatic systems of the person is in a compensated state.

(A5) In any of the systems denoted as (A2) through (A4), the functional zone assessment module may further include instructions stored in the memory, that when executed by the processor, perform the function of associating the person with a third of the plurality of different functional zones if at least two somatic systems of the person are in a compensated state.

(A6) Any of the systems denoted as (A2) through (A5) may further include a wellness care management module, including instructions stored in the memory, that when executed by the processor, perform the function of generating wellness care instructions based at least partially on which of the plurality of different functional zones is associated with the person.

(A7) In the system denoted as (A6), the wellness care management module may further include instructions stored in the memory, that when executed by the processor, perform the functions of (1) receiving additional personal data representing one or more physical attributes of the person after the person has received wellness care, (2) storing the additional personal data in the database to update the holome, and (3) associating one of the plurality of different functional zones with the person based at least partially on the updated holome.

(A8) Any of the systems denoted as (A1) through (A7) may further include a wellness score assessment module, including instructions stored in the memory, that when executed by the processor, perform the function of determining a wellness score for the person at least partially from the holome, the wellness score characterizing an improbability that the person will experience a physical ailment. The wellness score optionally characterizes an improbability that the person will experience at least one avoidable physical ailment.

(A9) Any of the systems denoted as (A1) through (A8) may further include a biometric diagnostic device operable to collect the personal data. The holome construction module may further include instructions stored in the memory, that when executed by the processor, perform the function of controlling the biometric diagnostic device to collect the personal data.

(A10) In the system denoted as (A9), the biometric diagnostic device may include one or more pieces of exercise equipment having biometric diagnostic capability.

(A11) Any of systems denoted as (A1) through (A10) may further include a web portal module, including instructions stored in the memory, that when executed by the processor, perform the function of limiting access to the system at least partially based on receipt of valid biometric authentication information from a user.

(A12) The system denoted as (A2) may further include a wellness care management module, including instructions stored in the memory, that when executed by the processor, perform the function of generating instructions for resolving a compensated state of the person, based at least partially on which of the plurality of different functional zones is associated with the person.

(B1) A method for evaluating dynamic wellness (vitality) of a person may include the following steps: (1) collecting, in a vitality evaluation computer, personal data representing one or more physical attributes of the person, (2) storing the personal data in a database of the vitality evaluation computer, and (3) evaluating, using a processor of the vitality evaluation computer, the personal data stored in the database to determine whether each of one or more somatic systems of the person are in a compensated state.

(B2) In the method denoted as (B1), the step of collecting may include collecting the personal data using a biometric diagnostic device communicatively coupled to the vitality evaluation computer.

(B3) The method denoted as (B2) may further include controlling the biometric diagnostic device, at least partially by the vitality evaluation computer, to collect the personal data.

(B4) In any of the methods denoted as (B1) through (B3), the step of collecting may include collecting the personal data using one or more pieces of exercise equipment communicatively to the vitality evaluation computer and having biometric diagnostic capability.

(B5) In any of the methods denoted as (B1) through (B4), the step of collecting may include receiving the personal data from a smart phone communicatively coupled to the vitality evaluation computer.

(B6) Any of the methods denoted as (B1) through (B5) may further include evaluating, using the processor of the vitality evaluation computer, the personal data stored in the database to determine a wellness score for the person, where the wellness score characterizes an improbability that the person will experience a physical ailment. The wellness score optionally characterizes an improbability that the person will experience at least one avoidable physical ailment.

(B7) The method denoted as (B6) may further include determining, using the processor of the vitality evaluation computer, a wellness risk management premium or health insurance premium discount for the person, at least partially based on the wellness score.

(B8) Either of the methods denoted as (B6) or (B7) may further include (1) outputting the wellness score to a wellness risk management premium determination computer communicatively coupled to the vitality evaluation computer, and (2) determining, using a processor of the wellness risk management premium determination computer, a health insurance premium discount for the person based at least partially on the wellness score.

(B9) Any of the methods denoted as (B6) through (B8) may further include outputting the wellness score from the vitality evaluation computer in exchange for receiving payment acknowledgement via an electronic commerce system communicatively coupled to the vitality evaluation computer.

(B10) Any of the methods denoted as (B1) through (B9) may further include associating, using the processor of the vitality evaluation computer, one of a plurality of different functional zones with the person based at least partially on a relationship between somatic systems of the person that are in a compensated state. Each of the plurality of different functional zones may represent a different relative wellness state of the person.

(B11) The method denoted as (B10) may further include associating, using the processor of the vitality evaluation computer, the person with a first of the plurality of different functional zones if each of the one or more somatic systems of the person are in an uncompensated state.

(B12) Either of the methods denoted as (B10) or (B11) may further include associating, using the processor of the vitality evaluation computer, the person with a second of the plurality of different functional zones if only one of the one or more somatic systems of the person are in a compensated state.

(B13) Any of the methods denoted as (B10) through (B12) may further include associating, using the processor of the vitality evaluation computer, the person with a third of the plurality of different functional zones if two or more somatic systems of the person are in a compensated state.

(B14) The method denoted as (B13) may further include outputting from the vitality evaluation computer a referral to a health care provider as a result of associating the person with the third of the plurality of different functional zones.

(B15) Any of the methods denoted as (B10) through (B14) may further include generating, using the processor of the vitality evaluation computer, wellness care instructions for the person at least partially based on which of the plurality of different functional zones is associated with the person.

(B16) The method denoted as (B15) may further include the following steps after the step of generating: (1) collecting, in the vitality evaluation computer, additional personal data representing one or more physical attributes of the person, (2) storing the additional personal data in the database of the vitality evaluation computer, and (3) associating, using the processor of the vitality evaluation computer, one of the plurality of different functional zones with the person based at least partially on the additional personal data stored in the database.

(B17) Any of the methods denoted as (B10) through (B16) may further include associating one of the plurality of different functional zones with the person in exchange for receiving payment via an electronic commerce system communicatively coupled to the vitality evaluation computer.

(B18) Any of the methods denoted as (B1) through (B17) may further include limiting access to the personal data stored in the database of the vitality evaluation computer based at least partially on receipt of valid biometric authentication information from a user.

Changes may be made in the above methods and systems without departing from the scope hereof. For example, the procedures performed to determine a person's functional Zone status may be varied. Therefore, the matter contained in the above description and shown in the accompanying drawings should be interpreted as illustrative and not in a limiting sense. The following claims are intended to cover generic and specific features described herein, as well as all statements of the scope of the present method and system, which, as a matter of language, might be said to fall there between.

What is claimed is:

1. A system for evaluating vitality of a person, comprising:
a processor;
a memory communicatively coupled to the processor;
a database, stored within the memory;
a holome construction module, including instructions stored in the memory, that when executed by the processor, perform the functions of:
receiving personal data representing one or more physical attributes of the person, and
storing the personal data as a holome in the database; and
a functional zone assessment module, including instructions stored in the memory, that when executed by the processor, perform the function of evaluating dynamics of the holome to determine whether each of one or more somatic systems of the person are in a compensated state.

2. The system of claim 1, the functional zone assessment module further including instructions stored in the memory, that when executed by the processor, perform the function of associating one of a plurality of different functional zones with the person based at least partially on a relationship between somatic systems of the person that are in a compensated state, each of the plurality of different functional zones representing a different relative wellness state of the person.

3. The system of claim 2, further comprising a wellness care management module, including instructions stored in the memory, that when executed by the processor, perform the function of generating instructions for resolving a compensated state of the person, based at least partially on which of the plurality of different functional zones is associated with the person.

4. The system of claim 2, the functional zone assessment module further including instructions stored in the memory, that when executed by the processor, perform the function of associating the person with a first of the plurality of different functional zones if each of the one or more somatic systems of the person are in an uncompensated state.

5. The system of claim 4, the functional zone assessment module further including instructions stored in the memory, that when executed by the processor, perform the function of associating the person with a second of the plurality of different functional zones if only one of the one or more somatic systems of the person is in a compensated state.

6. The system of claim 5, the functional zone assessment module further including instructions stored in the memory, that when executed by the processor, perform the function of associating the person with a third of the plurality of different functional zones if at least two somatic systems of the person are in a compensated state.

7. The system of claim 6, further comprising a wellness care management module, including instructions stored in the memory, that when executed by the processor, perform the function of generating wellness care instructions based at least partially on which of the plurality of different functional zones is associated with the person.

8. The system of claim 7, the wellness care management module further including instructions stored in the memory, that when executed by the processor, perform the functions of:
receiving additional personal data representing one or more physical attributes of the person after the person has received wellness care;
storing the additional personal data in the database to update the holome; and
associating one of the plurality of different functional zones with the person based at least partially on the updated holome.

9. The system of claim 1, further comprising a wellness score assessment module, including instructions stored in the memory, that when executed by the processor, perform the function of determining a wellness score for the person at least partially from the holome, the wellness score characterizing an improbability that the person will experience a physical ailment.

10. The system of claim 9, the wellness score characterizing an improbability that the person will experience at least one avoidable physical ailment.

11. The system of claim 1, further comprising:
a biometric diagnostic device operable to collect the personal data;
the holome construction module further including instructions stored in the memory, that when executed by the processor, perform the function of controlling the biometric diagnostic device to collect the personal data.

12. The system of claim 11, the biometric diagnostic device comprising one or more pieces of exercise equipment having biometric diagnostic capability.

13. The system of claim 1, further comprising a web portal module, including instructions stored in the memory, that when executed by the processor, perform the function of limiting access to the system at least partially based on receipt of valid biometric authentication information from a user.

14. A method for evaluating vitality of a person, comprising:
collecting, in a vitality evaluation computer, personal data representing one or more physical attributes of the person;
storing the personal data in a database of the vitality evaluation computer; and
evaluating, using a processor of the vitality evaluation computer, the personal data stored in the database to determine whether each of one or more somatic systems of the person are in a compensated state.

15. The method of claim 14, the step of collecting comprising collecting the personal data using a biometric diagnostic device communicatively coupled to the vitality evaluation computer.

16. The method of claim 15, further comprising controlling the biometric diagnostic device, at least partially by the vitality evaluation computer, to collect the personal data.

17. The method of claim 14, the step of collecting comprising collecting the personal data using one or more pieces of exercise equipment communicatively to the vitality evaluation computer and having biometric diagnostic capability.

18. The method of claim 14, the step of collecting comprising receiving the personal data from a smart phone communicatively coupled to the vitality evaluation computer.

19. The method of claim 14, further comprising evaluating, using the processor of the vitality evaluation computer, the personal data stored in the database to determine a wellness score for the person, the wellness score characterizing an improbability that the person will experience a physical ailment.

20. The method of claim 19, the wellness score characterizing an improbability that the person will experience at least one avoidable physical ailment.

21. The method of claim 19, further comprising determining, using the processor of the vitality evaluation computer, a wellness risk management premium or health insurance premium discount for the person, at least partially based on the wellness score.

22. The method of claim 19, further comprising:
outputting the wellness score to a wellness risk management premium determination computer communicatively coupled to the vitality evaluation computer; and
determining, using a processor of the wellness risk management premium determination computer, a wellness risk management premium for the person based at least partially on the wellness score.

23. The method of claim 19, further comprising outputting the wellness score from the vitality evaluation computer in exchange for receiving payment acknowledgement via an electronic commerce system communicatively coupled to the vitality evaluation computer.

24. The method of claim 14, further comprising associating, using the processor of the vitality evaluation computer, one of a plurality of different functional zones with the person based at least partially on a relationship between somatic systems of the person that are in a compensated state, each of the plurality of different functional zones representing a different relative wellness state of the person.

25. The method of claim 24, further comprising associating, using the processor of the vitality evaluation computer, the person with a first of the plurality of different functional zones if each of the one or more somatic systems of the person are in an uncompensated state.

26. The method of claim 25, further comprising associating, using the processor of the vitality evaluation computer, the person with a second of the plurality of different functional zones if only one of the one or more somatic systems of the person are in a compensated state.

27. The method of claim 26, further comprising associating, using the processor of the vitality evaluation computer, the person with a third of the plurality of different functional zones if two or more somatic systems of the person are in a compensated state.

28. The method of claims 27, further comprising outputting from the vitality evaluation computer a referral to a health care provider as a result of associating the person with the third of the plurality of different functional zones.

29. The method of claim 27, further comprising generating, using the processor of the vitality evaluation computer, wellness care instructions for the person at least partially based on which of the plurality of different functional zones is associated with the person.

30. The method of claim 29, further comprising the following steps after the step of generating:
collecting, in the vitality evaluation computer, additional personal data representing one or more physical attributes of the person;
storing the additional personal data in the database of the vitality evaluation computer; and
associating, using the processor of the vitality evaluation computer, one of the plurality of different functional zones with the person based at least partially on the additional personal data stored in the database.

31. The method of claim 24, further comprising associating one of the plurality of different functional zones with the person in exchange for receiving payment via an electronic commerce system communicatively coupled to the vitality evaluation computer.

32. The method of claim 14, further comprising limiting access to the personal data stored in the database of the vitality evaluation computer based at least partially on receipt of valid biometric authentication information from a user.

33. A method for determining a wellness risk management premium for a person, comprising:
receiving, in a wellness risk management premium determination computer, a wellness score characterizing an improbability that the person will experience a physical ailment; and
determining, using a processor of the wellness risk management premium determination computer, the wellness risk management premium for the person at least partially based on the wellness score.

34. The method of claim 33, the step of receiving comprising:
collecting, in a vitality evaluation computer, personal data representing one or more physical attributes of the person;
storing the personal data in a database of the vitality evaluation computer;
evaluating, using a processor of the vitality evaluation computer, the personal data stored in the database to determine the wellness score; and
outputting the wellness score to the wellness risk management premium determination computer.

35. The method of claim 34, further comprising receiving, via an electronic commerce subsystem communicatively coupled to the vitality evaluation computer, payment acknowledgement in exchange for the step of outputting.
